(19) United States
(12) Patent Application Publication
(10) Pub. No.: US 2026/0305737 A1
(43) Pub. Date: Oct. 8, 2026

(54) DEVICES, SYSTEMS, AND METHODS FOR ENHANCING A WEARABLE ARTICLE FOR USE WITH A STRETCHABLE CIRCUIT

(71) Applicant: Liquid Wire Inc., Portland, OR (US)

(72) Inventors: Jessica Jo-Yee Lim, Portland, OR (US); Jorge E. Carbo, JR., Portland, OR (US); Sharna Mozelle Donovan, Hillsboro, OR (US); Michael Scott Amos, Beaverton, OR (US)

(73) Assignee: Liquid Wire Inc., Portland, OR (US)

(21) Appl. No.: 19/573,224

(22) Filed: Mar. 20, 2026

Related U.S. Application Data
(63) Continuation of application No. PCT/US24/51743, filed on Oct. 17, 2024.
(60) Provisional application No. 63/591,075, filed on Oct. 17, 2023.

(51) Int. Cl. A41D 1/00; A41D 31/18

(52) U.S. Cl. CPC A41D 1/005; A41D 31/185

(57) ABSTRACT

A wearable article configured to be worn about an appendage of a user is disclosed herein. The wearable article can include a stretchable circuit comprising a core, wherein the core comprises a first modulus of elasticity. The first modulus of elasticity increases an elastic resistance of the wearable article, and wherein the increased elastic resistance causes the wearable article to translate along the appendage when in use. The wearable article can further include at least one structural support mechanically coupled to the wearable article, wherein the structural support generates a biasing force configured to compensate for the increased elastic resistance provided by the first modulus of elasticity, such that the biasing force reduces the translation of the wearable article along the appendage when in use.

### CROSS-REFERENCE TO RELATED APPLICATIONS

The present application is a continuation of PCT Application No. PCT/US24/51743, filed 17 Oct. 2024, which claims the benefit of priority from U.S. Provisional Patent Application No. 63/591,075, filed 17 Oct. 2023. All applications referenced herein are relevant to the subject matter disclosed herein and are hereby incorporated by reference in their entirety, regardless of the specific portion of the specification in which they are referenced.

### FIELD

The present disclosure is generally related to wearable electronics and, more particularly, is directed to new and improved garment designs that are optimized for use with stretchable circuits.

### SUMMARY

The following summary is provided to facilitate an understanding of some of the innovative features unique to the aspects disclosed herein and is not intended to be a full description. A full appreciation of the various aspects can be gained by taking the entire specification, claims, and abstract as a whole.

In various aspects, wearable article configured to be worn about an appendage of a user is disclosed. The wearable article can include a stretchable circuit comprising a core, wherein the core comprises a first modulus of elasticity. The first modulus of elasticity increases an elastic resistance of the wearable article, and wherein the increased elastic resistance causes the wearable article to translate along the appendage when in use. The wearable article can further include at least one structural support mechanically coupled to the wearable article, wherein the structural support generates a biasing force configured to compensate for the increased elastic resistance provided by the first modulus of elasticity, such that the biasing force reduces the translation of the wearable article along the appendage when in use.

In various aspects, a wearable article configured to be worn about an appendage of a user is disclosed. The wearable article can include a core mechanically coupled to a first portion of the wearable article, wherein the core includes a first modulus of elasticity, wherein the first modulus of elasticity increases an elastic resistance of the wearable article, and wherein the increased elastic resistance causes the wearable article to translate along the appendage when in use, and a second portion including a second modulus of elasticity, wherein the second modulus of elasticity generates a biasing force configured to compensate for the increased elastic resistance provided by the first modulus of elasticity, such that the biasing force reduces the translation of the wearable article along the appendage when in use.

In various aspects, a method of enhancing a wearable article for use with a stretchable circuit is disclosed. The method can include identifying a first modulus of elasticity, wherein the first modulus of elasticity is associated with the stretchable circuit, determining a second modulus of elasticity, mechanically coupling the stretchable circuit to a first portion of the wearable article, wherein the first modulus of elasticity increases an elastic resistance of the wearable article, wherein the increased elastic resistance causes the wearable article to translate along an appendage when in use, and structurally altering a second portion of the wearable article until the second portion includes the second modulus of elasticity, wherein the second modulus of elasticity generates a biasing force configured to compensate for the increased elastic resistance provided by the first modulus of elasticity, such that the biasing force reduces the translation of the wearable article along the appendage when in use.

These and other features and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

Various features of the aspects described herein are set forth with particularity in the appended claims. The various aspects, however, both as to organization and methods of operation, together with advantages thereof, may be understood in accordance with the following description taken in conjunction with the accompanying drawings as follows:

FIGS. 1A and 1B illustrate perspective views of a wearable article that has not been enhanced for use with a stretchable circuit before and after use, respectively;

FIG. 2 illustrates a flat, layering view of a wearable article that has been enhanced for use with a stretchable circuit, according to at least one non-limiting aspect of the present disclosure;

FIGS. 3A and 3B illustrate stretchable circuits configured for use with an enhanced wearable article, according to at least one non-limiting aspect of the present disclosure; and

FIG. 4 illustrates a perspective, assembly view of the wearable article of FIG. 2, according to at least one non-limiting aspect of the present disclosure;

FIGS. 5A and 5B illustrate perspective views of a wearable article that has been enhanced for use with a stretchable circuit before and after use, respectively; and

FIG. 6 illustrates a method of enhancing a wearable article for use with a stretchable circuit is depicted, according to at least one non-limiting aspect of the present disclosure.

Corresponding reference characters indicate corresponding parts throughout the several views. The exemplifications set out herein illustrate various aspects of the invention, in one form, and such exemplifications are not to be construed as limiting the scope of the invention in any manner.

### DETAILED DESCRIPTION

Numerous specific details are set forth to provide a thorough understanding of the overall structure, function, manufacture, and use of the aspects as described in the disclosure and illustrated in the accompanying drawings. Well-known operations, components, and elements have not been described in detail so as not to obscure the aspects described in the specification. The reader will understand that the aspects described and illustrated herein are non-limiting examples, and thus it can be appreciated that the specific structural and functional details disclosed herein may be representative and illustrative. Variations and changes thereto may be made without departing from the scope of the claims. Furthermore, it is to be understood that such terms as “forward”, “rearward”, “left”, “right”, “upwardly”, “downwardly”, and the like are words of convenience and are not to be construed as limiting terms. Furthermore, it is to be understood that such terms as “forward”, “rearward”, “left”, “right”, “upwardly”, “downwardly”, and the like are words of convenience and are not to be construed as limiting terms.

In the following description, like reference characters designate like or corresponding parts throughout the several views of the drawings. Also in the following description, it is to be understood that such terms as “forward”, “rearward”, “left”, “right”, “upwardly”, “downwardly”, and the like are words of convenience and are not to be construed as limiting terms.

A portion of the disclosure of this patent document contains material which is subject to copyright protection. The copyright owner has no objection to the facsimile reproduction by anyone of the patent disclosure, as it appears in the Patent and Trademark Office patent files or records, but otherwise reserves any and all copyrights disclosed herein.

Electronic circuits that are flexible and deformable have emerged as a means of innovating conventional electronics and introducing electronics into new products and applications. However, it would be beneficial for flexible electronic circuits to form a sealed, internal cavity, which can be filled with a compressible fluid. Such circuits could expand and contract in accordance with the selective insertion and/or removal of the fluid from the internal cavity. Moreover, the change in circuit geometry could lead to a subsequent change in electrical parameters generated across the inflatable circuit, which could be used to characterize a structural parameter or condition of the circuit, as desired. Indeed, inflatable circuits could provide numerous benefits for airbags, bladders, and/or cushions, which could be calibrated monitored, and even controlled based on measured electrical parameters. Accordingly, there is a need for devices, systems, and methods for making and using an inflatable circuit.

While certain electronic components typically have some inherent flexibility, such flexibility is typically constrained both in the amount the components can flex, their resilience in flexing, and the number of times the electronic components can flex before the electronic components deteriorate or break. Consequently, the utility of such electronic components in various environments may be limited, either by reliability or longevity or by the ability to function at all. Moreover, the lateral size of such components may result in additional stresses placed on the component. The use of conductive gel, however, can provide for electronic components that are flexible and deformable while maintaining resiliency. Moreover, the operational flexing, stretching, deforming, or other physical manipulation of a conductive trace formed from conductive gel may produce predictable, measurable changes in the electrical characteristics of the trace.

However, it shall be appreciated that stretchable circuits featuring traces composed of deformable conductors can decrease the modulus of elasticity of a wearable article, thereby adversely effecting the position of the wearable article about an appendage of a user. Worse, once the wearable article and thus, the stretchable circuit, is adversely positioned, it might adversely effect signals generated by the deformable conductor and reduce the efficacy of the wearable article for its intended application. Accordingly, there is a need for devices, systems, and methods for enhancing a garment for use with a stretchable circuit. As will be described herein, such devices, systems, and methods could alter the material characteristics of the wearable article and accommodate for any impacts to the modulus of elasticity caused by the stretchable circuit.

The present application is related to U.S. patent application Ser. No. 15/947,744, titled DEFORMABLE CONDUCTORS AND RELATED SENSORS, ANTENNAS AND MULTIPLEXED SYSTEMS, filed Apr. 6, 2018, and published as U.S. Patent Application Publication No. 2018/0247727 on Aug. 30, 2018, U.S. patent application Ser. No. 16/157,102, titled SENSORS WITH DEFORMABLE CONDUCTORS AND SELECTIVE DEFORMATION, filed Oct. 11, 2018, and published as U.S. Patent Application Publication No. 2019/0056277 on Feb. 21, 2019, U.S. patent application Ser. No. 16/885,854, titled CONTINUOUS INTERCONNECTS BETWEEN HETEROGENEOUS MATERIALS, filed May 28, 2020, and published as U.S. Patent Application Publication No. 2020/0381349 on Dec. 3, 2020, U.S. patent application Ser. No. 16/893,427, titled DEFORMABLE SENSORS WITH SELECTIVE RESTRAINT, filed Jun. 4, 2020, and published as U.S. Patent Application Publication No. 2020/0386630 on Dec. 3, 2020, U.S. patent application Ser. No. 17/192,725, titled DEFORMABLE INDUCTORS, filed Mar. 4, 2021, and published as U.S. Patent Application Publication No. 2021/0280482 on Sep. 9, 2021, and U.S. Provisional Patent Application No. 63/263,112, titled TWO DIMENSIONAL MOTION CAPTURE STRAIN GAUGE SENSOR, filed Oct. 10, 2021, the disclosures of which are hereby incorporated by reference in their entirety.

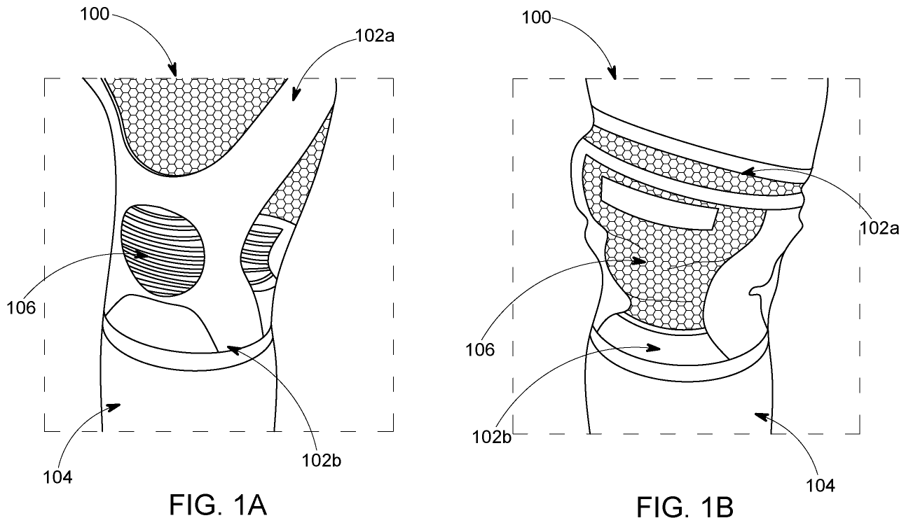

Referring now to FIGS. 1A and 1B, perspective views of a wearable article 100 that has not been enhanced for use with a stretchable circuit 106 are depicted before and after use, respectively. It shall be appreciated that, although the wearable article 100 of FIGS. 1A and 1B is configured to be worn about a specific appendage 104 (e.g., a leg) of a user, other wearable articles configured to be worn about any appendage of the user can be susceptible to the problem illustrated by FIGS. 1A and 1B. For example, although not visible in FIGS. 1A and 1B, the wearable article 100 can include a stretchable circuit 106 that is integrated into the wearable article 100 and configured to traverse from an upper portion 102ₐ of the wearable article, across a knee of the user, terminating at a lower portion 102_{b} of the wearable article 100.

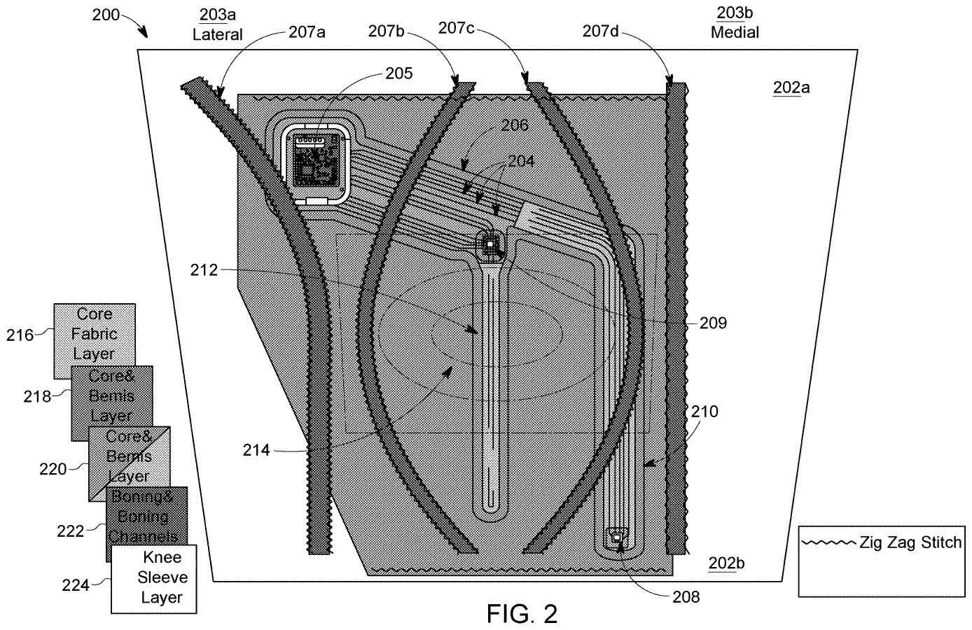

According to FIGS. 1A and 1B, the stretchable circuit 106 can include a stretchable circuit 106 the includes traces (see e.g., traces 204 in FIG. 2) formed from deformable conductors. It shall be appreciated that, while certain electronic components typically have some inherent flexibility, that flexibility is typically constrained both in the amount the components can flex, their resilience in flexing, and the number of times the electronic components can flex before the electronic components deteriorate or break. Moreover, electronics that have the ability to stretch, such as those comprising silver or other conductive inks, have insufficient durability and typically do not recover fully when subjected to elongation, resulting in ever-changing electrical characteristics until they fail completely.

The use of conductive gel for traces in a stretchable circuit, however, provides for electronic components that are flexible, extensible and deformable while maintaining resiliency. Moreover, the operational flexing, stretching, deforming, or other physical manipulation of a conductive trace formed from conductive gel may produce predictable, measurable changes in the electrical characteristics of the trace with little to no hysteresis upon returning to a relaxed state. By measuring the change in resistance or impedance of such a trace the change in length of the trace may be inferred. By combining the changes in lengths of multiple traces, the relative movement of points on a two-dimensional surface may be calculated. The relative movement of points in a three-dimensional space may be calculated and determined using two-dimensional displacement information if the points are disposed on a body that has constrained motion, for example, points located on limbs of a body that are interconnected by a joint. Therefore, according to the non-limiting aspect of FIGS. 1A and 1B, the stretchable circuit of the wearable article 100 can be constructed as disclosed in International Patent Application No. PCT/US2022/070850, titled HIGHLY SUSTAINABLE CIRCUITS AND METHODS FOR MAKING THEM, filed Feb. 25, 2022, and/or International Patent Application No. PCT/US2019/047731 titled STRUCTURES WITH DEFORMABLE CONDUCTORS, filed Aug. 22, 2019, the disclosures of which are hereby incorporated by reference in their entirety.

In summary, the traces of the stretchable circuit 106 can be constructed from a fluid-phase conductor. As used herein, the term “fluid-phase conductor” shall include any of the flexible, deformable conductors described herein and/or any of the flexible, deformable conductors described in any document incorporated by reference. Specifically, “fluid-phase conductors” are described in International Patent Application No. PCT/US2017/019762 titled LIQUID WIRE, which was filed on Feb. 27, 2017 and published on Sep. 8, 2017 as International Patent Publication No. WO2017/151523A1, and/or International Patent Application No. PCT/US2019/047731 titled STRUCTURES WITH DEFORMABLE CONDUCTORS, filed Aug. 22, 2019, the disclosures of which are hereby incorporated by reference in their entirety. According to some non-limiting aspects, each trace can include a variety of forms, such as a liquid, a paste, a gel, and/or a powder, amongst others that would enable the traces to have a deformable (e.g., soft, flexible, stretchable, bendable, elastic, flowable viscoelastic, Newtonian, non-Newtonian, etc.) quality. According to some non-limiting aspects, the deformable, conductive materials can include an electroactive material, such as deformable conductors produced from a conductive gel (e.g., a gallium indium alloy). The conductive gel can have a shear thinning composition and, according to some non-limiting aspects, can include a mixture of materials in a desired ratio. For example, according to one preferable non-limiting aspect, the conductive gel can include a weight percentage of a eutectic gallium alloy between 59.9% and 99.9% and a weight percentage of a gallium oxide between 0.1% and about 2.0%. Of course, the present disclosure contemplates other non-limiting aspects, featuring traces of varying forms and/or compositions to achieve the benefits disclosed herein.

The electrically conductive compositions can be characterized as conducting shear thinning gel compositions. The electrically conductive compositions described herein can also be characterized as compositions having the properties of a Bingham plastic. For example, the electrically conductive compositions can be viscoplastics, such that they are rigid and capable of forming and maintaining three-dimensional features characterized by height and width at low stresses but flow as viscous fluids at high stress. According to other non-limiting aspects, the low-shear viscosity of useful metal gel can be 10⁶ to 4×10⁷ Pa*s (1,000,000-40,000,000 Pascal seconds), wherein “low-shear” viscosity refers to a viscosity at rest (or sedimentation) conditions. The micro/nanostructure comprises oxide sheets that form a cross-linked structure, which may be achieved e.g. by mixing in a way that entrains air into the mixture, or by sonication that induces cavitation at the surface drawing in air to the mixture such that oxide formation in the cross-linked structures can be achieved.

It shall be appreciated that, by using stretchable circuits and deformable conductors, various sensors can be constructed that, when integrated into a wearable article 100—which, according to some non-limiting aspects can include a glove, a sleeve, a brace, a shirt, pants, shorts, or a sock, amongst other wearable articles—worn by a user, can generate varying electrical parameters (e.g., an inductance, a resistance, a voltage drop, a capacitance, and an electromagnetic field, etc.) that can be correlated to physical parameters (e.g., a strain, a stress, a pressure, a dimension, etc.). Accordingly, the wearable article 100 of FIGS. 1A and 1B can be used to generate highly accurate simulations of the user's motions while wearing the article. For example, the wearable article 100 can utilize the stretchable circuit 106 and its deformable conductors to function as a sensors (e.g., a strain sensor, etc.). Enabled by the deformable conductor, which is configured to move with the appendage 104, the wearable article 100 can actively and accurately monitor a user's motions without substantial electrical or physical degradation over thousands of strain cycles. Accordingly, continuous calibration is unnecessary and conversely, the stretchable circuit 106 can be used to calibrate conventional sensors (e.g., inertial measurement units (IMUs), etc.). In addition, parts of the circuit can be specifically configured and positioned to measure strain and thus, swelling in a particular location of the patient's appendage 104 (e.g., shin, etc.).

According to some non-limiting aspects, the stretchable circuit 106, itself, can include a multi-layer construction, including a substrate layer upon which the traces of the deformable conductor are deposited and covered with an encapsulation layer, which may include a flexible, stretchable material. For example, the multilayer construction can be similar to those disclosed in International Patent Application No. PCT/US2022/070850, titled HIGHLY SUSTAINABLE CIRCUITS AND METHODS FOR MAKING THEM, filed Feb. 25, 2022, and/or International Patent Application No. PCT/US2019/047731 titled STRUCTURES WITH DEFORMABLE CONDUCTORS, filed Aug. 22, 2019, the disclosures of which are hereby incorporated by reference in their entirety. For example, according to some non-limiting aspects, the multi-layer construction can include a substrate layer, a stencil-layer, and an encapsulation layer—in other non-limiting aspects, the substrates can include a two-layer construction (e.g., substrate layer, encapsulation layer, etc.) or even a single layer configured to accommodate the deformable traces. However, according to some non-limiting aspects, the primary material of the wearable article 100, itself, can include any of the aforementioned flexible and/or stretchable materials.

Any of the aforementioned layers can be constructed from any one of a number of polymers, such as, for example, thermoplastic polyurethane (TPU), high density polyethylene (HOPE), polyethylene terephthalate (PET), polyvinyl chloride (PVC), low density polyethylene (LDPE), polypropylene (PP) and/or polystyrene (PS), amongst others. However, according to other non-limiting aspects, any of the layers can be made from Epoxy-based materials, such as, for example, B-stage resin films. The Epoxy-based materials can be used as an additional discrete component to provide a self-adhesive surface for bonding electric components to any layer and/or for bonding layers to each other. In various other aspects, sustainability all layers, adhesives and encapsulants of any of the circuit assemblies disclosed herein can may be made from thermally activated adhesives, such as, for example, thermoplastic polyurethane (RU) adhesives (e.g., from Bemis or Framis). In yet other aspects, any of the layers, adhesives, and/or encapsulants of the circuit assembly can include thermoset adhesives such as, for example, silicones, acrylics, and any pressure sensitive adhesive of any chemistry, in the case of encapsulant materials, (e.g., encapsulates applied to terminals of an electric component), the concentration of thermoset material included in the encapsulant may be minimal, allowing the material to be readily recyclable. In one aspect, the circuit assemblies disclosed herein can include fillers including thermoset materials. Fillers including thermoset material may be reused in a “re-grind” format to strengthen, modify, or lower the cost of articles made from a similar thermosetting material.

It shall be appreciated that, in spite of the many benefits provided by the stretchable circuit 106 and its deformable conductors, the elastic nature of the stretchable circuit 106 can decrease the modulus of elasticity of the wearable article 100, thereby adversely effecting the position of the wearable article 100 about the appendage 104 of a user. In other words, the stretchable circuit 106 can increase the elastic load of the wearable article 100 and thus, more easily overcome frictional forces that would otherwise keep the wearable article 100 properly positioned about the appendage 104 during use.

For example, in reference to FIG. 1A, the wearable article 100 has been properly positioned about the appendage 104 of the user prior to use. Specifically, an upper portion 102ₐ of the wearable article 100 is properly positioned about an upper portion of the user's appendage 104 (e.g., the user's leg). As such, the upper portion 102ₐ of the wearable article 100 is properly positioned above a joint of the user's appendage 104 (e.g., the user's knee). A lower portion 102_{b} of the wearable article 100 is properly positioned about an upper portion of the user's lower appendage 104 (e.g., the user's leg). As such, the lower portion 102_{b} of the wearable article 100 is properly positioned below the joint of the user's appendage 104 (e.g., the user's knee).

However, according to the non-limiting aspect of FIG. 1B, the wearable article 100 has been used to monitor motions of the appendage 104. As such, the increased elastic load resulting from the integration of the stretchable circuit 106 into the wearable article 100 has overcome the frictional forces that originally kept the wearable article 100 properly positioned about the appendage 104, resulting in the wearable article 100 being improperly positioned about the user's appendage 104. Specifically, driven by the inclusion of the stretchable circuit 106, the upper portion 102ₐ of the wearable article 100 has succumbed to the increased elastic load of the wearable article 100 and overcome frictional forces. Thus, the upper portion 102ₐ of the wearable article 100 has slouched down the upper portion of the user's appendage 104, and is approaching the joint (e.g., the user's knee). Moreover, the materials of the wearable article 100 and the stretchable circuit 106—including the traces made from the deformable conductor—are improperly compressed. This can result in discomfort, as well as a distortion of signals generated by the traces of the stretchable circuit 106 and therefore, a largely inoperable device.

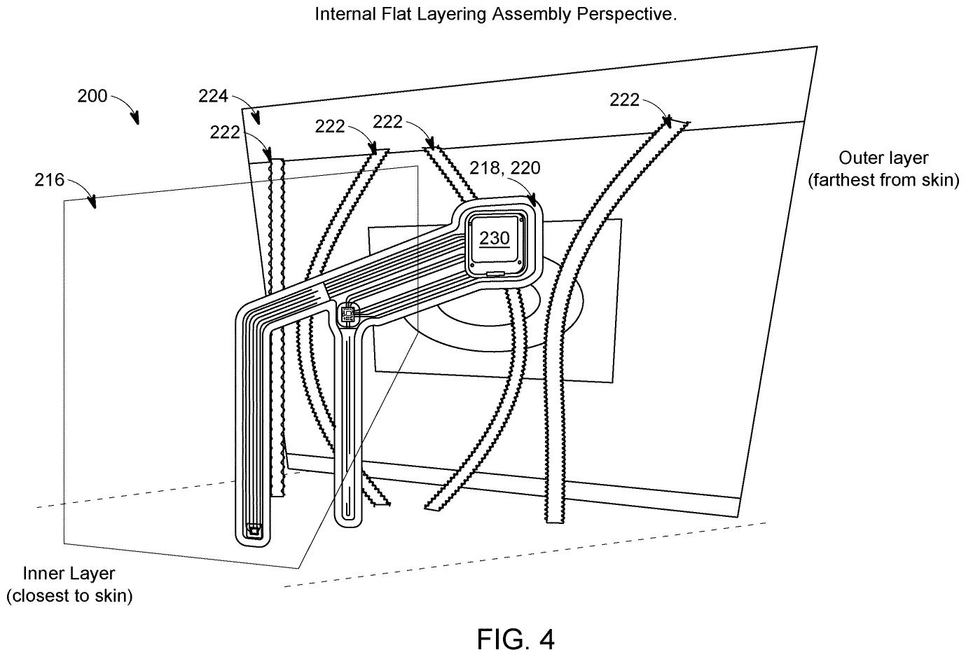

Referring now to FIG. 2, a flat, layering view of a wearable article 200 that has been enhanced for use with a stretchable circuit 206 is depicted in accordance with at least one non-limiting aspect of the present disclosure. According to the non-limiting aspect of FIG. 2, the wearable article 200 can be composed from an inner fabric layer 216, one or more core layers 218, 220, and/or an outer sleeve layer 224 mechanically coupled via any number of adhesives (e.g., those manufactured by Bemis), thermo-adhesive films and processes, stitching, and/or any suitable means of mechanical coupling, as will be described in further detail with reference to FIG. 4. However, according to other non-limiting aspects, any of the inner fabric layer 216, the one or more core layers 218, 220, and/or the outer sleeve layer 224 can be formed from a single, integral piece of material. For example, one or more core layers 218, 220 can function as a substrate layer of a stretchable circuit 206 that includes one or more traces 204 made from a deformable conductor. The stretchable circuit 206 and traces 204 can be constructed from any of the materials and/or constructions previously described in reference to the stretchable circuit 106 of FIGS. 1A and 1B. However, the features of the stretchable circuit 206 are depicted and will be described in further detail, in reference to the non-limiting aspect of FIG. 2. Of course, the features of the stretchable circuit 206 of FIG. 2 are non-limiting and the present disclosure contemplates other aspects with stretchable circuits of alternate configurations that vary in accordance with user preference and/or intended application. For example, although the stretchable circuit 206 configuration of FIG. 2 is intended to monitor motions of a user's knee via a knee brace, the configuration can be altered for different wearable articles configured to monitor different appendages of the user.

According to the non-limiting aspect of FIG. 2, the stretchable circuit 206 can include an integrated circuit 205, one or more analog-to-digital converters (ADC) 209, and/or one or more IMUs 208 electrically coupled via the one or more traces 204 made from a deformable conductor. For example, one or more of the traces 204 can be configured to function as a power and/or data bus line 210, configured to transmit electrical power and/or data to and from various components of the stretchable circuit 206. Additionally, one or more of the traces 204 can be configured to function as a sensor 212, configured to generate generate varying electrical parameters (e.g., an inductance, a resistance, a voltage drop, a capacitance, and an electromagnetic field, etc.) that can be correlated to physical parameters (e.g., a strain, a stress, a pressure, a dimension, etc.) associated with a motion of the appendage 104 (FIGS. 1A and 1B). However, according to some non-limiting aspects, the one or more of the traces 204 configured to function as a power and/or data bus line 210 can also be configured to function as a sensor and/or the one or more of the traces 204 configured to function as a sensor 212 can also be configured function as a power and/or data bus line. Regardless, it shall be appreciated that, as previously described, the traces 204 of the wearable article 200 can be used to generate highly accurate simulations of motions of an appendage 104 (FIG. 1) when worn. According to some non-limiting aspects, the wearable article 200 can further include a joint portion 214 substantially overlaying the joint of the wearer. The joint portion 214 may include elements constructed from foam or another biasing material configured to enhance the function of the sensor 212 at a particular location on the wearable article 200. Alternatively, the joint portion 214 may be made from a material having a higher stretch value than other portions of the wearable article 200 since the joint portion 214 may be subjected to the largest elongations when the wearer bends their appendage (at the joint).

According to some non-limiting aspects, the integrated circuit 205 can include one or more electronic components (e.g., a rechargeable power source, a control circuit, such as a microprocessor, a global positioning sensor (GPS), and/or a wireless transceiver, etc.). According to other non-limiting aspects, the integrated circuit 205 can include a cradle configured to mechanically and electrically couple an ancillary electronic component (e.g., a rechargeable power source, a control circuit, such as a microprocessor, a GPS, and/or a wireless transceiver, etc.) to the stretchable circuit 206, such as those disclosed in International Patent Application No. PCT/US2023/075870, titled DEVICES, SYSTEMS, AND METHODS FOR INTERACTING WITH & CALIBRATING A WEARABLE ARTICLE FEATURING CIRCUITS, filed Oct. 3, 2023, the disclosure of which is incorporated by reference herein. According to still other non-limiting aspects, a cradle can attach and ancillary electronic component (e.g., a rechargeable power source, a control circuit, such as a microprocessor, a GPS, and/or a wireless transceiver, etc.) directly to a substrate layer of the stretchable circuit 206, independent of the integrated circuit 205. Regardless, either the integrated circuit 205 and/or the electronic component, when mechanically and electrically coupled to the wearable article 200, can provide power and/or data to and from the stretchable circuit 206. Accordingly, it shall be appreciated that the integrated circuit 205 and an electronic component can provide the stretchable circuit 206—and therefore, the wearable article 200—with modular functionality.

In further reference to FIG. 2, one or more features configured to enhance the wearable article 200 for use with the stretchable circuit 206 by compensating for the increased elastic load presented by the stretchable circuit 206. For example, according to the non-limiting aspect of FIG. 2, the wearable article can include one or more structural supports 207_{a-d} configured to be inserted into one or more channels 222 stitched into the outer sleeve layer 224 and therefore, provide structural support to the wearable article 200 and compensate for the increased elastic load presented by the stretchable circuit 206. The one or more structural supports 207_{a-d} can be formed from a braided metal, such as steel or aluminum, for example. However, the present disclosure contemplates other materials including a variety of metals, alloys, and synthetic materials such as plastics. Notably, the materials can provide a certain spring constant while providing a sufficient resistance to axial compression, thereby enabling the one or more structural supports 207_{a-d} to bend with the wearable article 200, which promotes comfort, but prevents the inner fabric layer 216 and outer sleeve layer 224 from compressing in on themselves, as illustrated in FIG. 1B. It shall be appreciated that the one or more structural supports 207_{a-d} can provide a biasing force when mechanically coupled to the outer sleeve layer 224, and the biasing force can be greater than the resistance force of the wearable article 200 and the stretchable circuit 206, combined. In other words, the one or more structural supports 207_{a-d} can include a spring constant that is greater than a resistive force per unit of extension distance of the wearable article 200 and the stretchable circuit 206.

A first structural support 207ₐ and a second structural support 207_{d}, for example, can be positioned on a lateral side 203ₐ of the wearable article 200 and a medial side 203_{b} of the wearable article 200, respectively. According to the non-limiting aspect of FIG. 2, the second structural support 207_{d} can be relatively straight when viewed from the flat, layering view of FIG. 2. The spring constant ensures that the second structural support 207_{d} will flex when the appendage 104 (FIG. 1) is bent about the joint (e.g., the knee) and, because the second structural support 207_{d} is configured to resist axial compression, the second structural support 207_{d} will stretch an upper portion 202ₐ and lower portion 202_{b} of the wearable article 200, preventing the compression of FIG. 1B. The first structural support 207ₐ is similarly configured, relatively straight when viewed from the flat, layering view of FIG. 2. However, according to the non-limiting aspect of FIG. 2, the first structural support 207ₐ includes a lateral curve at the upper portion 202ₐ of the wearable article 200 to avoid structural interference with the integrated circuit 205.

Still referring to FIG. 2, a third structural support 207_{b} and a fourth structural support 207_{c} can be positioned about the joint (e.g., the knee) when the wearable article 200 is worn about the appendage 104 (FIG. 1) (e.g., the leg). According to the non-limiting aspect of FIG. 2, the third structural support 207_{b} and the fourth structural support 207_{c} can be bowed away from the joint (e.g., the knee) when viewed from the flat, layering view of FIG. 2. The spring constant ensures that the third and fourth structural support 207_{b, c} will straighten when the joint (e.g., the knee) of the appendage 104 (FIG. 1) (e.g., the leg) is bent and, because the third and fourth structural support 207_{b,d c} are configured to resist axial compression, the third and fourth structural support 207_{b,d c} will continue to stretch the upper portion 202ₐ and lower portion 202_{b} of the wearable article 200, preventing the compression of FIG. 1B. In other words, the one or more structural supports 207_{a-d} are configured to work in tandem to stretch the upper portion 202ₐ and the lower portion 202_{b} of the wearable article 200 throughout various motions, thereby preventing the compression of FIG. 1B, promoting comfort, and ensuring proper functionality of the stretchable circuit 206.

Although the non-limiting aspect of FIG. 2 discloses the use of one or more structural supports 207_{a-d} to enhance the wearable article 200 for use with a stretchable circuit 206, it shall be appreciated that the present disclosure further contemplates alternate means of enhancement. For example, according to some non-limiting aspects, the upper portion 202ₐ and lower portion 202_{b} of the wearable article 200 can be engineered to have a higher modulus of elasticity relative to the joint portion 214 of the wearable article 200, thereby increasing rigidity about the upper portion 202ₐ and lower portion 202_{b} of the wearable article 200, providing less flexibility and therefore, configuring the upper portion 202ₐ and lower portion 202_{b} of the wearable article 200 to function as anchor points. Contrarily, since the joint portion 214 of the wearable article 200 has a lower modulus of elasticity (or higher stretch) relative to the upper portion 202ₐ and lower portion 202_{b} of the wearable article 200, all flexions will occur about the joint portion 214 while the upper portion 202ₐ and lower portion 202_{b} ensure that the wearable article 200 remains properly positioned about the appendage 104 (FIG. 1). According to some non-limiting aspects, this may be accomplished by using a looser knit about the joint portion 214 relative to a tighter knit about the upper portion 202ₐ and lower portion 202_{b} of the outer sleeve layer 224 of the wearable article 200. Alternately or additionally, a back side of the wearable article 200 may be engineered to have a higher modulus of elasticity (or lower stretch) relative to a front side of the wearable article 200, such as the joint portion 214, thereby enabling flexions about the joint portion 214 but increasing rigidity on the backside of the wearable article 200. In other words, the back of the wearable article 200 can assist in keeping the upper portion 202ₐ and lower portion 202_{b} of the wearable article 200 properly positioned without compromising flexions of the stretchable circuit 206 about the joint portion 214.

For example, according to some non-limiting aspects, the material properties of the various layers and portions of the wearable article 200 can be measured in accordance with an industry standard, such as an ASTM (e.g., ASTM D6614, ASTM D3107, ASTM D2594, amongst others), depending on a fabric type implemented via the wearable article 200. However, according to some non-limiting aspects, the material properties of the various layers and portions of the wearable article 200 can be measured in accordance with ISO 13934-1, and other ISO standards. According to some non-limiting aspects, other material parameters, aside from the elastic modulus, can be calculated to characterize the stretchiness of various layers and portions of the wearable article 200, including Poisson's ratio and/or shear modulus, amongst others. In particular, the amount of stretch the various layers and portions have may be a useful parameter for comparison purposes. It should be appreciated that, when referring to the stretch of a particular portion or layer in relation to another portion or layer, indicating that one has a higher stretch value to the other means that the measured and/or calculated stretch using the appropriate test method yields a greater value for stretch. Similarly, indicating a lower stretch value means that the measured and/or calculated stretch using the appropriate test method yields a lower value for stretch for that portion or layer compared to another.

For example, Poisson's ratio can be used to describe how a the wearable article 200 changes its shape when it is stretched or compressed. It can be defined as the negative ratio of the lateral strain (e.g., the change in width or thickness) to the axial strain (e.g., the change in length) along the direction of an force applied by the appendage 104 (FIG. 1) as the wearable article 200 is used. For example, if the wearable article 200 is stretched by 10% in length and contracts by 5% in width, its Poisson's ratio is −0.5. A high Poisson's ratio means that the material tends to contract more when stretched, while a low Poisson's ratio means that the material tends to maintain its shape when stretched. Alternately, the shear modulus can be used to describe a measure of the rigidity or stiffness of the wearable article 200 when it is subjected to a shear force, which is a force that causes two parallel layers of the material to slide past each other. The shear modulus can be defined as the ratio of the shear stress (e.g., the force per unit area) to the shear strain (e.g., the angular displacement of the layers) in the plane of the force applied by the appendage 104 (FIG. 1) as the wearable article 200 is used. For example, if the wearable article 200 is twisted by 10 degrees and the shear stress is 100 kPa, its shear modulus is 10 kPa/degree. A high shear modulus means that the wearable article 200 resists deformation when twisted, while a low shear modulus means that the wearable article 200 easily deforms when twisted.

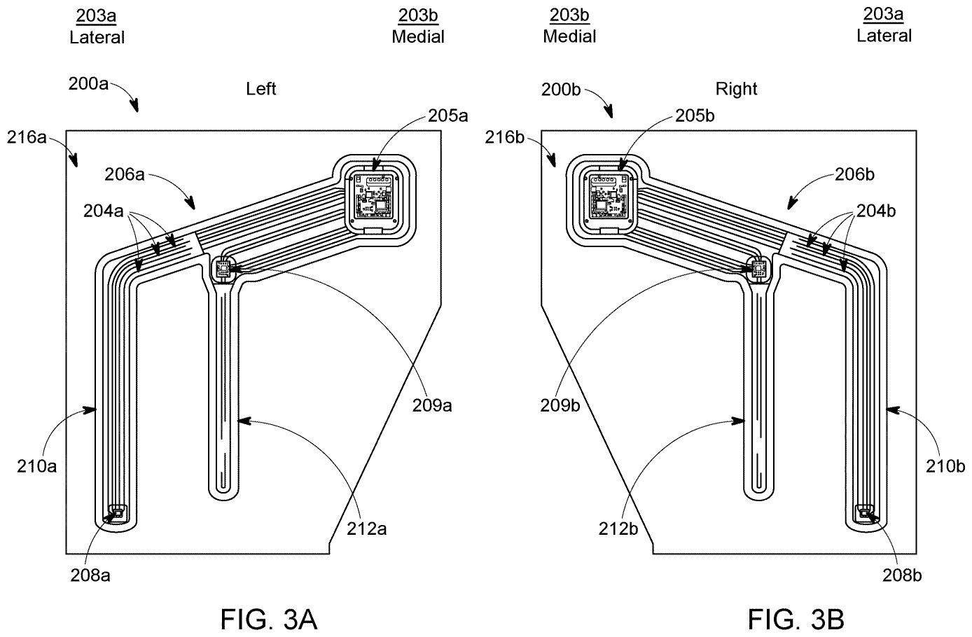

Referring now to FIGS. 3A and 3B, various stretchable circuits 206ₐ, 206_{b} configured for use with an enhanced wearable article are depicted according to at least one non-limiting aspect of the present disclosure. According to the non-limiting aspects of FIGS. 3A and 3B, the stretchable circuits 206ₐ, 206_{b} can include a construction and composition similar to the stretchable circuit 206 of FIG. 2. For example, as previously mentioned in reference to FIG. 2, and as will be described in further detail with reference to FIG. 4, each stretchable circuit 206ₐ, 206_{b} can be mechanically coupled to an inner fabric layer 216ₐ, 216_{b}. Additionally, each of the stretchable circuits 206ₐ, 206_{b} can include an integrated circuit 205ₐ, 205_{b}, one or more ADCs 209ₐ, 209_{b}, and/or one or more IMUs 208ₐ, 208_{b} electrically coupled via the one or more traces 204ₐ, 204_{b} made from a deformable conductor. Once again, one or more of the traces 204ₐ, 204_{b} can be configured to function as a power and/or data bus line 210ₐ, 210_{b}, configured to transmit electrical power and/or data to and from various components of the stretchable circuit 206ₐ, 206_{b}. Additionally, one or more of the traces 204ₐ, 204_{b} can be configured to function as a sensor 212ₐ, 212_{b} that generates varying electrical parameters (e.g., an inductance, a resistance, a voltage drop, a capacitance, and an electromagnetic field, etc.), which can be correlated to physical parameters (e.g., a strain, a stress, a pressure, a dimension, etc.) associated with a motion of the appendage 104 (FIGS. 1A and 1B).

However, according to the non-limiting aspect of FIG. 3A, a stretchable circuit 206_{b} can be similarly configured to the stretchable circuit 206 of FIG. 2, such that the stretchable circuit 206_{b} can be implemented to monitor motions of a left appendage 104 (e.g., a left leg). Accordingly, it shall be appreciated that the integrated circuit 205_{b} is positioned on a medial side 203_{b} of the wearable article 200_{b} and the data bus line 210_{b} is positioned on a lateral side 203ₐ of the wearable article 200_{b}. The sensor 212_{b}, however, is positioned to traverse across a joint (e.g., a knee) of the appendage 104 (FIG. 1) (e.g., leg). Similarly, the stretchable circuit 206ₐ of FIG. 3B can be inversely configured relative to the stretchable circuit 206 of FIG. 2, such that the stretchable circuit 206ₐ can be implemented to monitor motions of a right appendage 104 (FIG. 1) (e.g., a right leg). Accordingly, it shall be appreciated that the integrated circuit 205ₐ is positioned on a medial side 203_{b} of the wearable article 200ₐ and the data bus line 210ₐ is positioned on a lateral side 203ₐ of the wearable article 200ₐ. Once again, the sensor 212ₐ, however, should remain positioned to traverse across a joint (e.g., a knee) of the appendage 104 (FIG. 1) (e.g., leg).

It shall therefore be appreciated that the one or more channels 222 (FIG. 2) configured to receive the one or more structural supports 207_{a-d} (FIG. 2) should be positioned relative to each component of each stretchable circuit 206ₐ, 206_{b}, as depicted in FIG. 2. In other words, the stretchable circuit 206_{b} of FIG. 3B could benefit from one or more channels 222 (FIG. 2) positioned as depicted in FIG. 2, but the stretchable circuit 206ₐ of FIG. 3A could benefit from one or more channels 222 (FIG. 2) positioned inverse relative to the one or more channels 222 of FIG. 2.

Referring now to FIG. 4, a perspective, assembly view of the wearable article 200 of FIG. 2 is depicted in accordance with at least one non-limiting aspect of the present disclosure. As previously discussed, the wearable article 200 can be composed from an inner fabric layer 216, one or more core layers 218, 220, and/or an outer sleeve layer 224 mechanically coupled via any number of adhesives (e.g., those manufactured by Bemis), thermo-adhesive films and processes, stitching, and/or any suitable means of mechanical coupling, as will be described in further detail with reference to FIG. 4. According to the non-limiting aspect of FIG. 4, an exemplary assembly of the inner fabric layer 216, the one or more core layers 218, 220, and the outer sleeve layer 224 is depicted. For example, the inner fabric layer 216 can be positioned closest to the appendage 104 (FIG. 1) of the user. After the layup structure (e.g., including any of a substrate layer, a stencil layer, a deformable conductor, and/or an encapsulation layer, etc.) of the stretchable circuit 206 (FIG. 2) is constructed, the one or more core layers 218, 220 can be mechanically coupled or bonded to the inner fabric layer 216.

In further reference to the non-limiting aspect of FIG. 4, the one or more channels 222 configured to receive the one or more structural supports 207_{a-d} (FIG. 2) can be stitched (or otherwise coupled) to the outer sleeve layer 224. Of course, according to other non-limiting aspects, the one or more channels 222 can be integral to the outer sleeve layer 224 to reduce the amount of additional required. The one or more structural supports 207_{a-d} (FIG. 2) can be inserted into the one or more channels 222 and, once inserted, the outer sleeve layer 224 can be mechanically coupled to the inner fabric layer 216 and the one or more core layers 218, 220, including the stretchable circuit 206 (FIG. 2). According to non-limiting aspects wherein the stretchable circuit 206 (FIG. 2) further includes a modular component 230 configured to be mechanically and electrically coupled to the integrated circuit 205 (FIG. 2), it may be beneficial to include an opening in the outer sleeve layer 224, whereby a user can access a pocket positioned between the outer sleeve layer 224 and the stretchable circuit 206 (FIG. 2), so the modular component 230 can be selectively coupled to and from the integrated circuit 205 (FIG. 2). Of course, according to other non-limiting aspects, any of the inner fabric layer 216, the one or more core layers 218, 220, and/or the outer sleeve layer 224 can be integrally formed from a single piece of material.

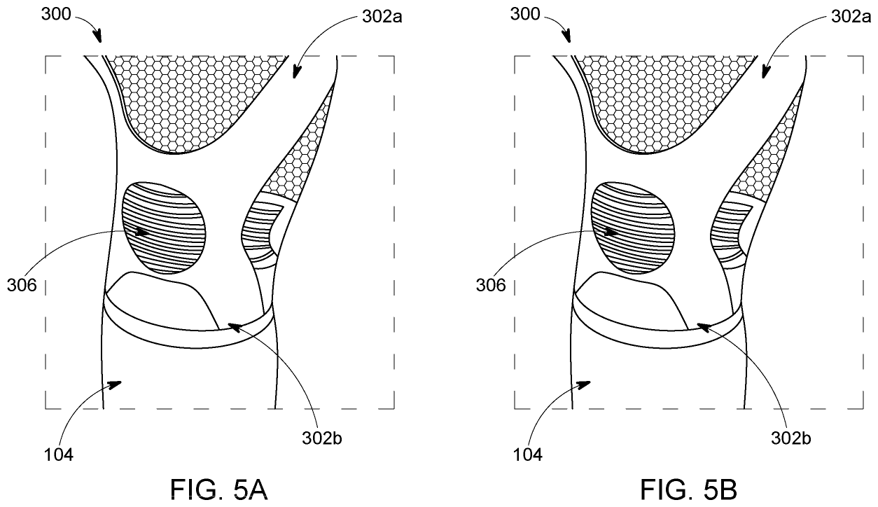

Referring now to FIGS. 5A and 5B, perspective views of a wearable article 300 that has been enhanced for use with a stretchable circuit 306 before and after use are depicted in accordance with at least one non-limiting aspect of the present disclosure, respectively. For example, the wearable article 300 of FIGS. 5A and 5B can be configured similar to the wearable article 200 of FIG. 2. According to FIG. 5A, the wearable article 300 has been properly positioned about the appendage 104 of the user prior to use. Specifically, an upper portion 302ₐ of the wearable article 300 is properly positioned about an upper portion of the user's appendage 104 (e.g., the user's leg). As such, the upper portion 302ₐ of the wearable article 300 is properly positioned above a joint of the user's appendage 104 (e.g., the user's knee). A lower portion 302_{b} of the wearable article 300 is properly positioned about an upper portion of the user's lower appendage 104 (e.g., the user's leg). As such, the lower portion 302_{b} of the wearable article 100 is properly positioned below the joint of the user's appendage 104 (e.g., the user's knee).

According to the non-limiting aspect of FIG. 5B, the wearable article 300 has been used to monitor motions of the appendage 104. However, unlike the wearable article 100 of FIGS. 1A and 1B, the wearable article 300 of FIGS. 5A and 5B has been enhanced for use with a stretchable circuit 306. As such, the increased elastic load resulting from the integration of the stretchable circuit 306 into the wearable article 300 has been offset and thus, could not overcome the frictional forces that originally kept the wearable article 300 properly positioned about the appendage 104. Accordingly, after use, the wearable article 300 remains properly positioned about the user's appendage 104. Specifically, the upper portion 302ₐ of the wearable article 100 has not succumbed to the increased elastic load of the wearable article 300—driven by the inclusion of the stretchable circuit 306—and thus, has not overcome frictional forces and is not slouched. Moreover, the materials of the wearable article 300 and the stretchable circuit 306—including the traces made from the deformable conductor—are not improperly compressed. This can promote comfort, as well as a preservation of signal integrity of the stretchable circuit 306.

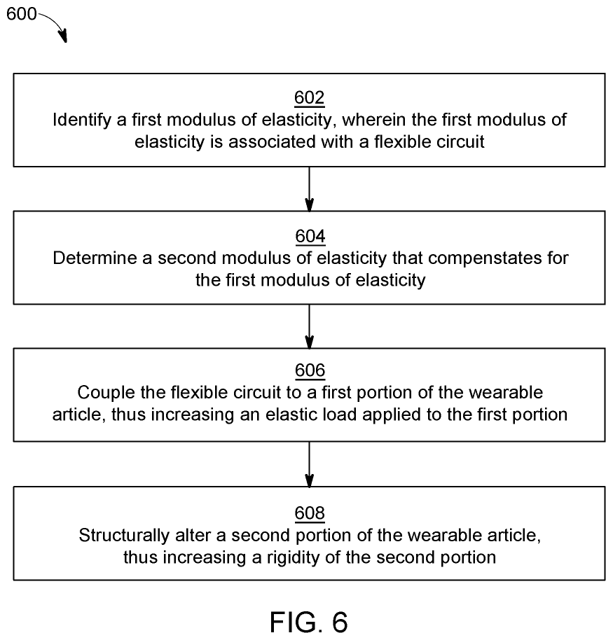

Referring now to FIG. 6, a method 600 of enhancing a wearable article for use with a stretchable circuit is depicted in accordance with at least one non-limiting aspect of the present disclosure. According to the non-limiting aspect of FIG. 6, the method 600 can include identifying 602 a first modulus of elasticity, wherein the first modulus of elasticity is associated with the stretchable circuit. The method 600 can further include determining 604 a second modulus of elasticity, wherein the second modulus of elasticity is configured to compensate for the first modulus of elasticity. In further reference to FIG. 6, the method 600 can include mechanically coupling 606 the stretchable circuit to a first portion of the wearable article. For example, according to some non-limiting aspects, the first portion may include a front of the wearable article or may correspond to a joint of the appendage. As discussed herein, the first modulus of elasticity can increase an elastic load applied to the first portion of the wearable article and thus, adversely effect a position of the wearable article about an appendage of a user. Accordingly, the method 600 can further include structurally altering 608 a second portion of the wearable article until the second portion includes the second modulus of elasticity. For example, the second portion may include an upper portion, a lower portion, or a back portion of the wearable article. As discussed herein, the second modulus of elasticity can increase a rigidity of the second portion of the wearable article, thereby compensating for the increased elastic load applied to the first portion of the wearable article and improving a position of the wearable article about an appendage of a user.

According to some non-limiting aspects of the present disclosure, structurally altering 608 the second portion of the wearable article can include mechanically coupling a plurality of structural supports to the wearable article. According to other non-limiting aspects of the present disclosure, structurally altering 608 the second portion of the wearable article can include increasing the stretch value of the second portion, e.g., by tightening a knit of the second portion. According to still other non-limiting aspects of the present disclosure, structurally altering 608 the second portion of the wearable article can include both mechanically coupling a plurality of structural supports to the wearable article and reducing the stretch value of the second portion of the wearable article.

Since the inventive principles of this patent disclosure can be modified in arrangement and detail without departing from the inventive concepts, such changes and modifications are considered to fall within the scope of the following claims. The use of terms such as first and second are for purposes of differentiating different components and do not necessarily imply the presence of more than one component.

Various aspects of the subject matter described herein are set out in the following numbered clauses:
Clause 1. A wearable article configured to be worn about an appendage of a user, the wearable article including a stretchable circuit including a core, wherein the core includes a first modulus of elasticity, wherein the first modulus of elasticity increases an elastic resistance of the wearable article, and wherein the increased elastic resistance causes the wearable article to translate along the appendage when in use, and at least one structural support mechanically coupled to the wearable article, wherein the structural support generates a biasing force configured to compensate for the increased elastic resistance provided by the first modulus of elasticity, such that the biasing force reduces the translation of the wearable article along the appendage when in use.
Clause 2. The wearable article according to clause 1, wherein elastic resistance generates a resistive force when the wearable is stretched, and wherein the biasing force has a value that is greater than the resistive force.
Clause 3. The wearable article according to either of clauses 1 or 2, further including an outer sleeve layer including at least one channel, wherein the at least one structural support is inserted within the at least one channel.
Clause 4. The wearable article according to any of clauses 1-3, further including an inner fabric layer, wherein the core of the stretchable circuit is mechanically coupled to the inner fabric layer.
Clause 5. The wearable article according to any of clauses 1-4, wherein the outer sleeve layer is mechanically coupled to the inner fabric layer, and wherein the stretchable circuit is positioned intermediate the inner fabric layer and the outer sleeve layer.
Clause 6. The wearable article according to any of clauses 1-5, wherein the at least one structural support is positioned intermediate the stretchable circuit and the outer sleeve layer.
Clause 7. The wearable article according to any of clauses 1-6, wherein the outer sleeve includes an upper portion including a third modulus of elasticity, and a lower portion including a fifth modulus of elasticity, wherein the third modulus of elasticity and the fifth modulus of elasticity generate a second and third biasing force, wherein each of the second and third biasing force are configured to further compensate for the increased elastic resistance provided by the first modulus of elasticity, such that the second and third biasing forces further reduce the translation of the wearable article along the appendage when in use.
Clause 8. The wearable article according to any of clauses 1-7, wherein the outer sleeve further includes a joint portion positioned between the upper portion and the lower portion, wherein the joint portion includes a looser knit relative to the upper portion and the lower portion of the outer sleeve, and wherein the looser knit is configured to further compensate for the increased elastic resistance provided by the first modulus of elasticity.
Clause 9. The wearable article according to any of clauses 1-8, wherein the outer sleeve includes a back portion including a sixth modulus of elasticity that generates a fourth biasing force configured to further compensate for the increased elastic resistance provided by the first modulus of elasticity, and a front portion including a seventh modulus of elasticity that reduces the elastic resistance of the wearable article, such that the fourth biasing force and the seventh modulus of elasticity further reduce the translation of the wearable article along the appendage when in use.
Clause 10. The wearable article according to any of clauses 1-9, wherein the wearable article undergoes a flexion when in use, and the at least one structural support provides the biasing force when the flexion occurs, and wherein the at least one structural support further provides a resistance to axial compression configured to reduce bunching of the wearable article about the appendage when the flexion has ceased.
Clause 11. A wearable article configured to be worn about an appendage of a user, the wearable article including a stretchable circuit including a core mechanically coupled to a first portion of the wearable article, wherein the core includes a first modulus of elasticity, wherein the first modulus of elasticity increases an elastic resistance of the wearable article, and wherein the increased elastic resistance causes the wearable article to translate along the appendage when in use, and a second portion including a second modulus of elasticity, wherein the second modulus of elasticity generates a biasing force configured to compensate for the increased elastic resistance provided by the first modulus of elasticity, such that the biasing force reduces the translation of the wearable article along the appendage when in use.
Clause 12. The wearable article according to clause 11, wherein elastic resistance generates a resistive force when the wearable is stretched, and wherein the biasing force has a value that is greater than the resistive force.
Clause 13. The wearable article according to either of clauses 11 or 12, wherein the first portion is a front portion of the wearable article, wherein the second portion is a back portion of the wearable article.
Clause 14. The wearable article according to any of clauses 11-13, wherein the first portion is an joint portion of the wearable article, wherein the second portion is an upper portion of the wearable article.
Clause 15. The wearable article according to any of clauses 11-14, further including a lower portion including a third modulus of elasticity that generates a second biasing force configured to compensate for the increased elastic resistance provided by the first modulus of elasticity, such that the second biasing force further reduces the translation of the wearable article along the appendage when in use.
Clause 16. The wearable article according to any of clauses 11-15, wherein the first portion includes a looser knit relative to the second portion of the wearable article.
Clause 17. The wearable article according to any of clauses 11-16, further including at least one structural support mechanically coupled to the wearable article, wherein the at least one structural support includes a fourth modulus of elasticity that generates a third biasing force configured to compensate for the increased elastic resistance provided by the first modulus of elasticity, such that the third biasing force further reduces the translation of the wearable article along the appendage when in use.
Clause 18. The wearable article according to any of clauses 11-17, further including an outer sleeve layer including at least one channel, wherein the at least one structural support is inserted within the at least one channel.
Clause 19. The wearable article according to any of clauses 11-18, further including an inner fabric layer, wherein the core of the stretchable circuit is mechanically coupled to the inner fabric layer.
Clause 20. The wearable article according to any of clauses 11-19, wherein the outer sleeve layer is mechanically coupled to the inner fabric layer, and wherein the stretchable circuit is positioned intermediate the inner fabric layer and the outer sleeve layer.
Clause 21. The wearable article according to any of clauses 11-20, wherein the at least one structural support is positioned intermediate the stretchable circuit and the outer sleeve layer.
Clause 22. A method of enhancing a wearable article for use with a stretchable circuit, the method including identifying a first modulus of elasticity, wherein the first modulus of elasticity is associated with the stretchable circuit, determining a second modulus of elasticity, mechanically coupling the stretchable circuit to a first portion of the wearable article, wherein the first modulus of elasticity increases an elastic resistance of the wearable article, wherein the increased elastic resistance causes the wearable article to translate along an appendage when in use, and structurally altering a second portion of the wearable article until the second portion includes the second modulus of elasticity, wherein the second modulus of elasticity generates a biasing force configured to compensate for the increased elastic resistance provided by the first modulus of elasticity, such that the biasing force reduces the translation of the wearable article along the appendage when in use.
Clause 23. The method according to clause 22, wherein structurally altering the second portion of the wearable article includes mechanically coupling at least one structural support to the wearable article.
Clause 24. The method according to either of clauses 22 or 23, wherein structurally altering the second portion of the wearable article includes tightening a knit of the second portion of the wearable article relative to a knit of the second portion of the wearable article.
Clause 25: The devices disclosed herein.
Clause 26: The systems disclosed herein.
Clause 27: The methods disclosed herein.

All patents, patent applications, publications, or other disclosure material mentioned herein, are hereby incorporated by reference in their entirety as if each individual reference was expressly incorporated by reference respectively. All references, and any material, or portion thereof, that are said to be incorporated by reference herein are incorporated herein only to the extent that the incorporated material does not conflict with existing definitions, statements, or other disclosure material set forth in this disclosure. As such, and to the extent necessary, the disclosure as set forth herein supersedes any conflicting material incorporated herein by reference and the disclosure expressly set forth in the present application controls.

The present invention has been described with reference to various exemplary and illustrative aspects. The aspects described herein are understood as providing illustrative features of varying detail of various aspects of the disclosed invention; and therefore, unless otherwise specified, it is to be understood that, to the extent possible, one or more features, elements, components, constituents, ingredients, structures, modules, and/or aspects of the disclosed aspects may be combined, separated, interchanged, and/or rearranged with or relative to one or more other features, elements, components, constituents, ingredients, structures, modules, and/or aspects of the disclosed aspects without departing from the scope of the disclosed invention. Accordingly, it will be recognized by persons having ordinary skill in the art that various substitutions, modifications or combinations of any of the exemplary aspects may be made without departing from the scope of the invention. In addition, persons skilled in the art will recognize, or be able to ascertain using no more than routine experimentation, many equivalents to the various aspects of the invention described herein upon review of this specification. Thus, the invention is not limited by the description of the various aspects, but rather by the claims.

Those skilled in the art will recognize that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B”.

With respect to the appended claims, those skilled in the art will appreciate that recited operations therein may generally be performed in any order. Also, although claim recitations are presented in a sequence(s), it should be understood that the various operations may be performed in other orders than those which are described, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

It is worthy to note that any reference to “one aspect,” “an aspect,” “an exemplification,” “one exemplification,” and the like means that a particular feature, structure, or characteristic described in connection with the aspect is included in at least one aspect. Thus, appearances of the phrases “in one aspect,” “in an aspect,” “in an exemplification,” and “in one exemplification” in various places throughout the specification are not necessarily all referring to the same aspect. Furthermore, the particular features, structures or characteristics may be combined in any suitable manner in one or more aspects.

As used herein, the singular form of “a”, “an”, and “the” include the plural references unless the context clearly dictates otherwise.

Directional phrases used herein, such as, for example and without limitation, top, bottom, left, right, lower, upper, front, back, and variations thereof, shall relate to the orientation of the elements shown in the accompanying drawing and are not limiting upon the claims unless otherwise expressly stated.

The terms “about” or “approximately” as used in the present disclosure, unless otherwise specified, means an acceptable error for a particular value as determined by one of ordinary skill in the art, which depends in part on how the value is measured or determined. In certain aspects, the term “about” or “approximately” means within 1, 2, 3, or 4 standard deviations. In certain aspects, the term “about” or “approximately” means within 50%, 200%, 105%, 100%, 9%, 8%, 7%, 6%, 5%, 4%, 3%, 2%, 1%, 0.5%, or 0.05% of a given value or range.

In this specification, unless otherwise indicated, all numerical parameters are to be understood as being prefaced and modified in all instances by the term “about,” in which the numerical parameters possess the inherent variability characteristic of the underlying measurement techniques used to determine the numerical value of the parameter. At the very least, and not as an attempt to limit the application of the doctrine of equivalents to the scope of the claims, each numerical parameter described herein should at least be construed in light of the number of reported significant digits and by applying ordinary rounding techniques.

Any numerical range recited herein includes all sub-ranges subsumed within the recited range. For example, a range of “1 to 100” includes all sub-ranges between (and including) the recited minimum value of 1 and the recited maximum value of 100, that is, having a minimum value equal to or greater than 1 and a maximum value equal to or less than 100. Also, all ranges recited herein are inclusive of the end points of the recited ranges. For example, a range of “1 to 100” includes the end points 1 and 100. Any maximum numerical limitation recited in this specification is intended to include all lower numerical limitations subsumed therein, and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited. All such ranges are inherently described in this specification.

Any patent application, patent, non-patent publication, or other disclosure material referred to in this specification and/or listed in any Application Data Sheet is incorporated by reference herein, to the extent that the incorporated materials is not inconsistent herewith. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a system that “comprises,” “has,” “includes” or “contains” one or more elements possesses those one or more elements, but is not limited to possessing only those one or more elements. Likewise, an element of a system, device, or apparatus that “comprises,” “has,” “includes” or “contains” one or more features possesses those one or more features, but is not limited to possessing only those one or more features.

Instructions used to program logic to perform various disclosed aspects can be stored within a memory in the system, such as dynamic random access memory (DRAM), cache, flash memory, or other storage. Furthermore, the instructions can be distributed via a network or by way of other computer readable media. Thus a machine-readable medium may include any mechanism for storing or transmitting information in a form readable by a machine (e.g., a computer), but is not limited to, floppy diskettes, optical disks, compact disc, read-only memory (CD-ROMs), and magneto-optical disks, read-only memory (ROMs), random access memory (RAM), erasable programmable read-only memory (EPROM), electrically erasable programmable read-only memory (EEPROM), magnetic or optical cards, flash memory, or a tangible, machine-readable storage used in the transmission of information over the Internet via electrical, optical, acoustical or other forms of propagated signals (e.g., carrier waves, infrared signals, digital signals, etc.). Accordingly, the non-transitory computer-readable medium includes any type of tangible machine-readable medium suitable for storing or transmitting electronic instructions or information in a form readable by a machine (e.g., a computer).

As used in any aspect herein, any reference to a processor or microprocessor can be substituted for any “control circuit,” which may refer to, for example, hardwired circuitry, programmable circuitry (e.g., a computer processor including one or more individual instruction processing cores, processing unit, processor, microcontroller, microcontroller unit, controller, digital signal processor (DSP), programmable logic device (PLD), programmable logic array (PLA), or field programmable gate array (FPGA)), state machine circuitry, firmware that stores instructions executed by programmable circuitry, and any combination thereof. The control circuit may, collectively or individually, be embodied as circuitry that forms part of a larger system, for example, an integrated circuit (IC), an application-specific integrated circuit (ASIC), a system on-chip (SoC), desktop computers, laptop computers, tablet computers, servers, smart phones, etc. Accordingly, as used herein “control circuit” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

As used in any aspect herein, the term “logic” may refer to an app, software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices.

As used in any aspect herein, the terms “component,” “system,” “module” and the like can refer to a computer-related entity, either hardware, a combination of hardware and software, software, or software in execution.

Unless specifically stated otherwise as apparent from the foregoing disclosure, it is appreciated that, throughout the foregoing disclosure, discussions using terms such as “processing,” “computing,” “calculating,” “determining,” “displaying,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

One or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components and/or inactive-state components and/or standby-state components, unless context requires otherwise.

What is claimed is:

1. A wearable article configured to be worn about an appendage of a user, the wearable article comprising:
a stretchable circuit comprising a core, wherein the core comprises a first modulus of elasticity, wherein the first modulus of elasticity increases an elastic resistance of the wearable article, and wherein the increased elastic resistance causes the wearable article to translate along the appendage when in use; and
at least one structural support mechanically coupled to the wearable article, wherein the structural support generates a biasing force configured to compensate for the increased elastic resistance provided by the first modulus of elasticity, such that the biasing force reduces the translation of the wearable article along the appendage when in use.

2. The wearable article of claim 1, wherein elastic resistance generates a resistive force when the wearable is stretched, and wherein the biasing force has a value that is greater than the resistive force.

3. The wearable article of claim 1, further comprising an outer sleeve layer comprising at least one channel, wherein the at least one structural support is inserted within the at least one channel.

4. The wearable article of claim 3, further comprising an inner fabric layer, wherein the core of the stretchable circuit is mechanically coupled to the inner fabric layer.

5. The wearable article of claim 4, wherein the outer sleeve layer is mechanically coupled to the inner fabric layer, and wherein the stretchable circuit is positioned intermediate the inner fabric layer and the outer sleeve layer.

6. The wearable article of claim 5, wherein the at least one structural support is positioned intermediate the stretchable circuit and the outer sleeve layer.

7. The wearable article of claim 3, wherein the outer sleeve comprises:
a joint portion configured to overlay a joint of the user and having a first stretch value;
an upper portion located above the joint portion having a second stretch value;
a lower portion located below the joint portion and having a third stretch value, and wherein each of the second stretch value and the third stretch value are greater than the first stretch value.

8. The wearable article of claim 7, wherein the wherein the joint portion stretch value is selected to further compensate for the increased elastic resistance provided by the first modulus of elasticity.

9. The wearable article of claim 3, wherein the outer sleeve comprises:
a back portion comprising a fourth stretch value; and
a front portion comprising a fifth stretch value; and
wherein the fourth stretch value is smaller than the fifth stretch value, such that the translation of the wearable article along the appendage is further reduced when in use.

10. The wearable article of claim 1, wherein the wearable article undergoes a flexion when in use, and the at least one structural support provides the biasing force when the flexion occurs, and wherein the at least one structural support further provides a resistance to axial compression configured to reduce bunching of the wearable article about the appendage when the flexion has ceased.

11. A wearable article configured to be worn about an appendage of a user, the wearable article comprising:
a stretchable circuit comprising a core mechanically coupled to a first portion of the wearable article, wherein the core comprises a first modulus of elasticity, wherein the first modulus of elasticity increases an elastic resistance of a portion of the wearable article, and wherein the increased elastic resistance causes the wearable article to translate along the appendage when in use; and
a second portion configured to have a second modulus of elasticity greater than the first modulus of elasticity, such that translation of the wearable article along the appendage of the user is reduced when in use.

12. The wearable article of claim 1, wherein elastic resistance generates a resistive force when the wearable is stretched, and wherein the second modulus of elasticity is selected to reduce bunching of the wearable article caused by the resistive force.

13. The wearable article of claim 11, wherein the first portion is a front portion of the wearable article, wherein the second portion is a back portion of the wearable article.

14. The wearable article of claim 11, wherein the first portion is a joint portion of the wearable article configured to overlay a joint of the user, and wherein the second portion is an upper portion of the wearable article located above the joint portion.

15. The wearable article of claim 14, further comprising a lower portion comprising a third modulus of elasticity greater than the first modulus of elasticity, such that translation of the wearable article along the appendage of the user is further reduced when in use.

16. The wearable article of claim 11, wherein the first portion comprises a higher stretch value relative to the second portion of the wearable article.

17. The wearable article of claim 11, further comprising at least one structural support mechanically coupled to the wearable article, wherein the at least one structural support comprises generates a biasing force configured to compensate for the increased elastic resistance provided by the first modulus of elasticity, such that the third biasing force further reduces the translation of the wearable article along the appendage when in use.

18. The wearable article of claim 17, further comprising an outer sleeve layer comprising at least one channel, wherein the at least one structural support is inserted within the at least one channel.

19. The wearable article of claim 18, further comprising an inner fabric layer, wherein the core of the stretchable circuit is mechanically coupled to the inner fabric layer.

20. The wearable article of claim 19, wherein the outer sleeve layer is mechanically coupled to the inner fabric layer, and wherein the stretchable circuit is positioned intermediate the inner fabric layer and the outer sleeve layer.

21. The wearable article of claim 19, wherein the at least one structural support is positioned intermediate the stretchable circuit and the outer sleeve layer.

22. A method of enhancing a wearable article for use with a stretchable circuit, the method comprising:
identifying a first modulus of elasticity, wherein the first modulus of elasticity is associated with the stretchable circuit;
determining a second modulus of elasticity;
mechanically coupling the stretchable circuit to a first portion of the wearable article, wherein the first modulus of elasticity increases an elastic resistance of the wearable article, wherein the increased elastic resistance causes the wearable article to translate along an appendage when in use; and
structurally altering a second portion of the wearable article until the second portion comprises the second modulus of elasticity, wherein the second modulus of elasticity is configured to compensate for the increased elastic resistance provided by the first modulus of elasticity, such that translation of the wearable article along the appendage is reduced when in use.

23. The method of claim 22, wherein structurally altering the second portion of the wearable article comprises mechanically coupling at least one structural support to the wearable article.

24. The method of claim 22, wherein structurally altering the second portion of the wearable article comprises reducing the stretch value of the second portion of the wearable article relative to the stretch value of the second portion of the wearable article.
